(19) United States
(12) Patent Application Publication
(10) Pub. No.: US 2007/0037971 A1
(43) Pub. Date: Feb. 15, 2007

(54) PROCESS FOR DESILYLATION OF CARBAPENEM INTERMEDIATES

(76) Inventors: Hashim Nizar Poovanathil Nagoor Meeran, Kerala (IN); Bishwa Prakash Rai, Uttar Pradesh (IN); Shailendra Kumar Singh, Gurgaon (IN); Mohan Prasad, Gurgaon (IN)

Correspondence Address: RANBAXY INC., 600 COLLEGE ROAD EAST, SUITE 2100, PRINCETON, NJ, 08540, US

(21) Appl. No.: 11/461,062

(22) Filed: Jul. 31, 2006

(30) Foreign Application Priority Data
Jul. 29, 2005 (IN) 2017/DEL/2005

(51) Int. Cl. C07D 487/04; C07D 205/02

(52) U.S. Cl. 540/200; 540/350

(57) ABSTRACT

Provided are processes for desilylating carbapenem intermediates. In particular, such desilylation reactions are done in the presence of one or more desilylating agents selected from acetyl chloride, silica chloride, thionyl chloride, oxalyl chloride or mixtures thereof.

### FIELD OF THE INVENTION

Provided are process for desilylation of carbapenem intermediates. In particular, such desilylation reactions are done in the presence of one or more desilylating agents selected from acetyl chloride, silica chloride, thionyl chloride, oxalyl chloride or mixtures thereof.

### BACKGROUND OF THE INVENTION

Carbapenem compounds are known for their broad and potent antibacterial activity. A large number of derivatives have been prepared and investigated for clinical efficacy. Imipenem, faropenem, meropenem, ertapenem and doripenem are some of the carbapenem antibiotics available in the market for treating various bacterial infections.

Silyl protected intermediates of Formula I or analogues thereof are useful in preparing carbapenem antibiotics, 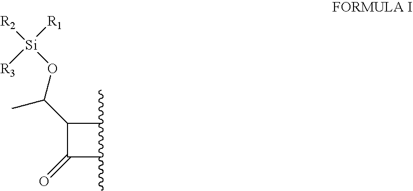
wherein R₁, R₂ and R₃ are same or different and are each C₁₋₅ alkyl.

Silyl protecting groups of compounds of Formula I or its analogues must eventually be removed during the preparation of carbapenem antibiotics. U.S. Pat. No. 4,683,296 discloses a desilylation process for carbapenem intermediates of Formula I and related compounds by using aqueous hydrochloric acid. Similarly U.S. Pat. No. 5,340,927 describes a desilylation process using methanesulfonic acid. A similar process is disclosed in U.S. Pat. No. 4,783,453, where desilylation is carried out using potassium phosphate.

The prior art methods involve the use of strong reaction conditions and excess quantity of desilylating agents. Desilylating methods that involve the use of mineral or sulfonic acids also require a considerable amount of base for neutralization. Carbapenem ring systems are sensitive to such acidic or basic conditions, resulting in reduced yield and increased impurity level.

In view of the above, there remains a need for novel processes for desilylating silyl-protected intermediates with higher yields and higher purity levels.

### SUMMARY OF THE INVENTION

Provided herein are efficient methods for desilylating carbapenem intermediates using one or more of an acetyl chloride, silica chloride, oxalyl chloride, thionyl chloride or mixtures thereof. The methods described herein utilize only a catalytic quantity of these chloride compounds and does not require an excessive amount of base for neutralization. Thus, the methods described herein provide facile, eco-friendly and high yielding desilylation with improved purity.

Thus in one aspect, provided are processes for preparing a compound of Formula II 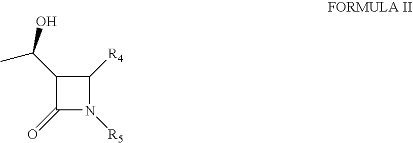
comprising:
desilylating a compound of Formula IV, 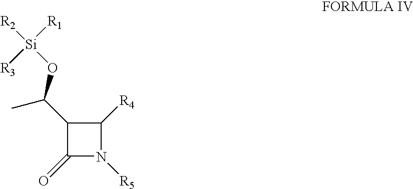
   in the presence of one or more desilylating agents selected from acetyl chloride, silica chloride, thionyl chloride, oxalyl chloride or a mixture thereof to form a compound of Formula II,
wherein
   P₁ can be hydrogen or a carboxyl protecting group;

P₂ can be hydrogen or an amino protecting group;
R₁, R₂ and R₃ are same or different and each independently can be C₁₋₅ alkyl;
R₄ can be selected from 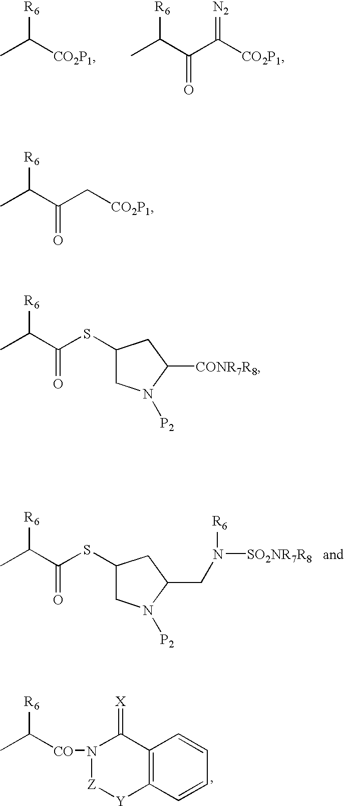
R₅ can be hydrogen,
or R₄ and R₅ can be joined together to form a resultant compound of Formula III; 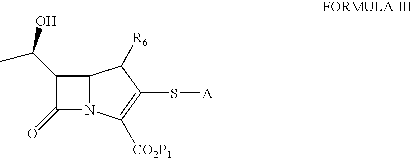
R₆ can be hydrogen or C₁₋₅ alkyl;
R₇ and R₈ are same or different and each independently can be hydrogen, C₁₋₅ alkyl, substituted or unsubstituted aryl, or substituted or unsubstituted heteroaryl;
X can be oxygen atom or sulfur atom;
Y can be oxygen atom, sulfur atom, substituted or unsubstituted methylene group or an imino group;
Z can be a substituted or unsubstituted methylene group; and
A can be selected from

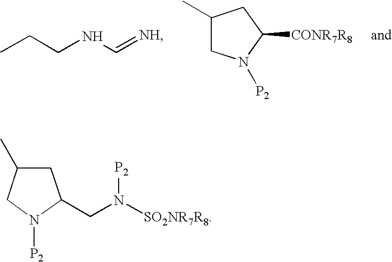

The processes can include one or more of the following embodiments. For example, the processes can be carried out in the presence of acetyl chloride, silica chloride, thionyl chloride, or oxalyl chloride. In another embodiment, the processes can be carried out in the presence of one or more organic solvents selected from one or more of C₁₋₅ alkanols, aromatic hydrocarbons, halogenated hydrocarbons, ketones, esters, ethers or mixtures thereof. Preferably, the one or more organic solvents can be one or more C₁₋₅ alkanols.

In another embodiment, the compound of Formula II can be isolated from the reaction mixture by layer separation or filtration. Layer separation can comprise adding a mixture of one or more miscible organic solvents and water after the compound of Formula IV is desilylated to form a layered mixture. In yet another embodiment, the layered mixture can be neutralized by adding one or more bases. Suitable bases can be selected from one or more of metal hydroxides, metal carbonates, metal oxides, amines or mixtures thereof. In another embodiment, the processes can be carried out at a temperature of about −20° C. to about 50° C.

### DETAILED DESCRIPTION OF THE INVENTION

The term “protecting group,” as used herein, refers to protecting groups typically used and known in the art and blocks carboxyl or amino groups while reactions are carried out at other sites of the molecule. Examples of a carboxyl protecting group include, but not limited to, optionally substituted C₁-C₈ alkyl, optionally substituted C₃-C₈ alkenyl, optionally substituted C₇-C₁₉ aralkyl, optionally substituted C₆-C₁₂ aryl, optionally substituted C₁-C₁₂ amino, optionally substituted C₃-C₁₂ hydrocarbonated silyl, optionally substituted C₃-C₁₂ hydrocarbonated stannyl, and pharmaceutically active ester forming groups. Examples of amino protecting groups include, but not limited to, lower alkylsilyl groups, lower alkoxymethyl groups, aralkyl groups, acetyl groups, lower alkoxycarbonyl groups, alkenyloxycarbonyl groups and aralkyloxycarbonyl groups.

Provided herein are processes for preparing a compound of Formula II 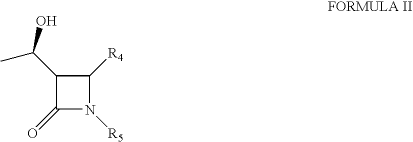
comprising:

desilylating a compound of Formula IV, 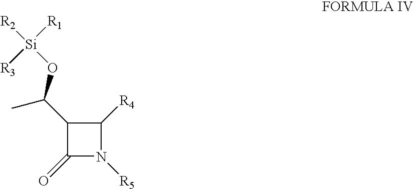
in the presence of one or more desilylating agents selected from acetyl chloride, silica chloride, thionyl chloride, oxalyl chloride or a mixture thereof to form a compound of Formula II, wherein
P₁ is hydrogen or a carboxyl protecting group;
P₂ is hydrogen or an amino protecting group;
R₁, R₂ and R₃ are same or different and are each independently C₁₋₅ alkyl;
R₄ is selected from 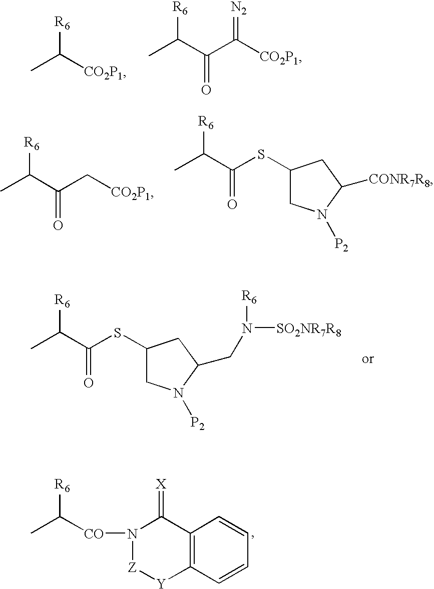
R₅ is hydrogen,
or R₄ and R₅ are joined together so as to form a resultant compound of Formula III; 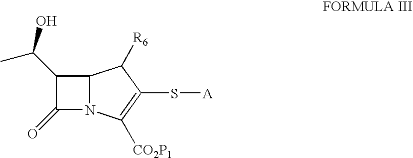
R₆ is hydrogen or C₁₋₅ alkyl;
R₇ and R₈ are same or different and are hydrogen, C₁₋₅ alkyl, substituted or unsubstituted aryl, or substituted or unsubstituted heteroaryl;
X is oxygen atom or sulfur atom;
Y is oxygen atom, sulfur atom, substituted or unsubstituted methylene group or an imino group;
Z is a substituted or unsubstituted methylene group; and
A is selected from

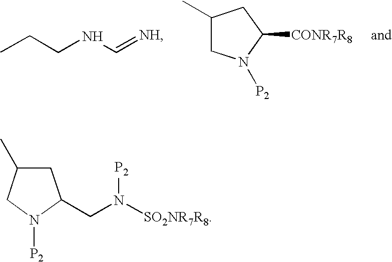

Compounds of Formula IV can be prepared by any of the methods known in the art. For example, the compounds of Formula IV can be prepared by methods disclosed in U.S. Pat. Nos. 6,011,150, 5,371,214, 4,918,184, 4,683,296, 5,340,927 and 4,783,453. each of which are incorporated by reference in its entirety. Compounds of Formula IV can be desilylated in the presence of one or more desilylating agents selected acetyl chloride or silica chloride or thionyl chloride, oxalyl chloride or mixtures thereof. The desilylation reaction can also be carried out in one or more organic solvents. Suitable organic solvents include, for example, one or more of C₁₋₅ alkanols, aromatic hydrocarbons, halogenated hydrocarbons, ketones, esters, ethers or mixtures thereof. The desilylation reaction can be carried out at a temperature from about −20° C. to about 50° C., preferably from about 15° C. to about 35° C. The desilylated compound can be isolated from the reaction mixture by conventional methods, for example, by layer separation or by filtration. In embodiments where the desilylated compounds are isolated by layer separation, the desilylation reaction mixture can be combined with a mixture of one or more water-immiscible solvents and water. Suitable water immiscible organic solvents can be selected from dichloromethane, n-hexane, toluene, cyclohexane, carbon tetrachloride, chloroform, 1,2-dichloroethane, heptane, diethyl ether, petroleum ether or mixtures thereof. The reaction mixture can be optionally neutralized to adjust the pH to about 4.5 to about 8 prior to isolating the desilylated compound. Neutralization can be carried out by the addition of one or more bases to the reaction mixture. Suitable bases can be selected from metal hydroxides, metal carbonates, metal oxides and amines. Preferred bases include, for example, sodium bicarbonate, sodium carbonate, sodium hydroxide, calcium carbonate and calcium hydroxide. Bases may be added as an aqueous solution. After neutralization, the compound of Formula II can be isolated from the reaction mixture by concentrating the organic layer. Compounds of Formula II can be optionally further purified by crystallization or chromatography.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Example 1

### Preparation of Compound of Formula IIa:

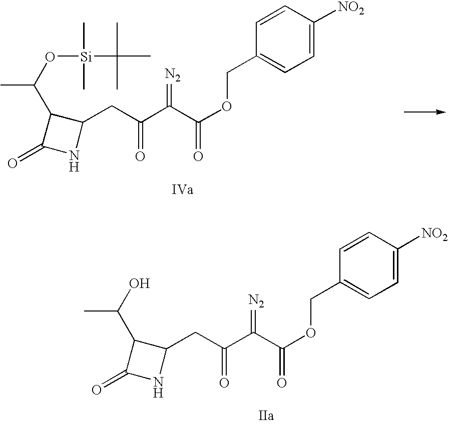

Acetyl chloride (2.7 g) was added to a mixture of a compound of Formula IVa (50 g) in toluene (500 ml) and methanol (100 ml). The reaction mixture was stirred for 2 hours at 20° C. to 25° C. The reaction was monitored by thin layer chromatography and a solid was separated by filtration. The separated solid was washed with methanol and dried to yield a compound of Formula IIa.

Yield: 36 g

HPLC Purity: 98%

¹H NMR: 1.29 (d, 3H), 2.85 (dd, 1H), 3.2 (m, 2H), 3.93 (m, 1H), 4.1 (m, 2H), 5.37 (s, 2H), 7.56 (d, 2H), 8.24 (d, 2H)

### Example 2

### Preparation of Compound of Formula IIb:

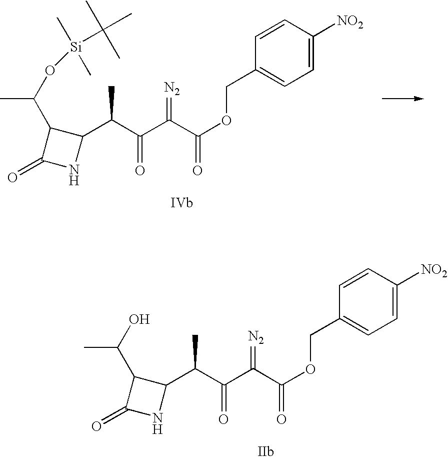

A compound of Formula IVb (100 g) was suspended in methanol (300 ml) at about 25° C. Acetyl chloride (3.1 g) was added to the suspension and stirred for 2 hours. After completion of the reaction, the reaction mixture (homogeneous solution) was poured into a mixture of dichloromethane (1 L) and water (700 ml), and the pH was adjusted to 7.0 by the addition of aqueous sodium bicarbonate (5% w/v) solution at about 25° C. The dichloromethane layer was separated, concentrated under vacuum and the residue was crystallized with toluene to yield the compound of Formula IIb.

Yield: 74 g

HPLC Purity: 99%

¹H NMR (CDCl₃): 8.28 (d, 2H), 7.54 (d. 2H), 6.16 (1H, NH), 5.36 (s, 2H), 4.13 (m, 1H), 3.86 (m, 2H), 3.78 (m, 1H), 2.91 (m, 1H), 1.30 (d, 3H), 1.22(d, 3H)

### Example 3

### Preparation of Compound of Formula IIa:

Using the procedure set forth in Example 1, acetyl chloride was replaced with thionyl chloride (4.1 g) to obtain a compound of Formula IIa. Thus, thionyl chloride (4.1 g) was added to a mixture of a compound of Formula IVa (50 g) in toluene (500 ml) and methanol (100 ml). The reaction mixture was stirred for 2 hours at 20° C. to 25° C. The reaction was monitored by thin layer chromatography and a solid was separated by filtration. The separated solid was washed with methanol and dried to yield a compound of Formula IIa.

Yield: 35 g

HPLC Purity: 97%

### Example 4

### Preparation of Compound of Formula IIb:

Using the procedure set forth in Example 2, acetyl chloride was replaced with silica chloride (25 g) to obtain a compound of Formula IIb. Thus, a compound of Formula IVb (100 g) was suspended in methanol (300 ml) at about 25° C. Silica chloride (25 g) was added to the suspension and stirred for 2 hours. After completion of the reaction, the reaction mixture (homogeneous solution) was poured into a mixture of dichloromethane (1 L) and water (700 ml), and the pH was adjusted to 7.0 by the addition of aqueous sodium bicarbonate (5% w/v) solution at about 25° C. The dichloromethane layer was separated, concentrated under vacuum and the residue was crystallized with toluene to yield the compound of Formula IIb.

Yield: 73 g

HPLC Purity: 99%

### Example 5

### Preparation of Compound of Formula IIb:

Using the procedure set forth in Example 2, acetyl chloride was replaced with thionyl chloride (4.7 g) to obtain a compound of Formula IIb. Thus, a compound of Formula IVb (100 g) was suspended in methanol (300 ml) at about 25° C. Thionyl chloride (4.7 g) was added to the suspension and stirred for 2 hours. After completion of the reaction, the reaction mixture (homogeneous solution) was poured into a mixture of dichloromethane (1 L) and water (700 ml), and the pH was adjusted to 7.0 by the addition of aqueous sodium bicarbonate (5% w/v) solution at about 25° C. The dichloromethane layer was separated, concentrated under vacuum and the residue was crystallized with toluene to yield the compound of Formula IIb.

Yield: 70 g

HPLC Purity: 99%

### Example 6

### Preparation of Compound of Formula IIc:

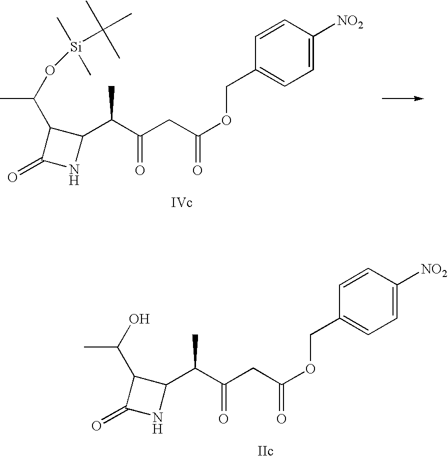

A compound of Formula IVc (20 g) was suspended in methanol (60 ml) at a temperature of about 25° C. Acetyl chloride (0.65 g) was added to the suspension so obtained and stirred for 2 hours. After completion of the reaction, the reaction mixture (homogeneous solution) was poured into a mixture of dichloromethane (1 L) and water (700 ml), and the pH was adjusted to 7.0 by the addition of aqueous sodium bicarbonate (5% w/v) solution at about 25° C. The dichloromethane layer was separated, concentrated under vacuum and the residue was crystallized with a mixture of ethyl acetate and hexane (1:1) to yield the compound of Formula IIc.

Yield: 12.7 g

HPLC Purity: 99%

¹H NMR (CDCl₃): 8.23 (d, 2H), 7.54 (d. 2H), 6.1 (1H, NH), 5.31 (s, 2H), 4.14 (m, 1H), 3.81 (m, 1H), 3.66 (s, 2H), 2.93 (m, 1H), 2.45 (m, 1H), 1.29 (d, 3H), 1.25 (d, 3H)

### Example 7

### Preparation of Compound of Formula IId:

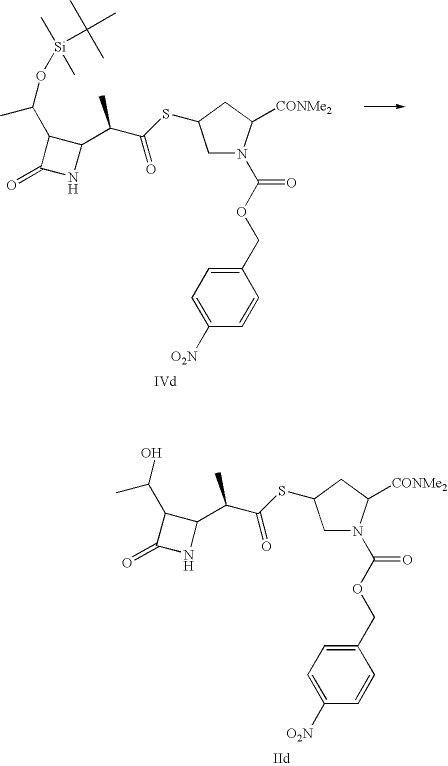

(2R)-2-[3-(1-{[tert-Butyl(dimethyl)silyl]oxy}ethyl)-4-oxoazetidin-2-yl]propanoic acid (10 g) was suspended in dichloromethane (100 ml) and cooled to −15° C. Ethyl chloroformate (4.4 g) was added to the suspension, followed by the addition of triethylamine (4.3 g) at −15° C. to −10° C. and the reaction mixture was stirred for 60 minutes. 4-Nitrobenzyl 2-[(dimethylamino)carbonyl]-4-mercaptopyrrolidine-1-carboxylate (11.7 g) was added to the reaction mixture at −10° C. and stirred for 60 minutes. Water (50 ml) was added to the reaction mixture and the pH was adjusted to 7.0 by the addition of aqueous sodium bicarbonate (2% w/v) solution at about 25° C. followed by washing with water (50 ml). The organic layer was separated and concentrated under vacuum to obtain a solid residue containing the compound of Formula IVd. The residue was dissolved in methanol (30 ml), followed by the addition of acetyl chloride (0.65 g). The reaction mixture was stirred for 60 minutes at 20° C. to 25° C. and subsequently poured into a mixture of dichloromethane (100 ml) and water (100 ml), followed by adjusting the pH to 5.0. The dichloromethane layer was separated and concentrated to yield the compound of Formula IId.

Yield: 7.0 g

Purity by HPLC: 95%

¹H NMR (CDCl₃): 8.21 (d, 2H), 7.50 (d, 2H), 6.09 (br, NH), 5.21 (s, 2H, OCH2), 4.25 (br, 1H), 4.13 (m, 2H), 4.01 (m, 2H), 3.77 (m, 1H), 3.51 (m, 2H), 2.99 (s, 3H), 2.93 (s, 3H), 2.70-3.0 (m, 2H), 1.90 (m, 1H), 1.31 (s, 3H), 1.27 (s, 3H)

### Example 8

### Preparation of Compound of Formula IId:

Using the procedure set forth in Example 7, acetyl chloride was replaced with silica chloride (2 g) to obtain IId. Thus, (2R)-2-[3-(1-{[tert-Butyl(dimethyl)silyl]oxy}ethyl)-4-oxoazetidin-2-yl]propanoic acid (10 g) was suspended in dichloromethane (100 ml) and cooled to −15° C. Ethyl chloroformate (4.4 g) was added to the suspension, followed by the addition of triethylamine (4.3 g) at −15° C. to −10° C. and the reaction mixture was stirred for 60 minutes. 4-Nitrobenzyl 2-[(dimethylamino)carbonyl]-4-mercaptopyrrolidine-1-carboxylate (11.7 g) was added to the reaction mixture at −10° C. and stirred for 60 minutes. Water (50 ml) was added to the reaction mixture and the pH was adjusted to 7.0 by the addition of aqueous sodium bicarbonate (5% w/v) solution at about 25° C. followed by washing with water (50 ml). The organic layer was separated and concentrated under vacuum to obtain a solid residue containing the compound of Formula IVd. The residue was dissolved in methanol (30 ml), followed by the addition of silica chloride (2 g). The reaction mixture was stirred for 60 minutes at 20° C. to 25° C. and subsequently poured into a mixture of dichloromethane (100 ml) and water (100 ml), followed by adjusting the pH to 5.0. The dichloromethane

Yield: 6.5 g

### Example 9

### Preparation of Compound of Formula IIe:

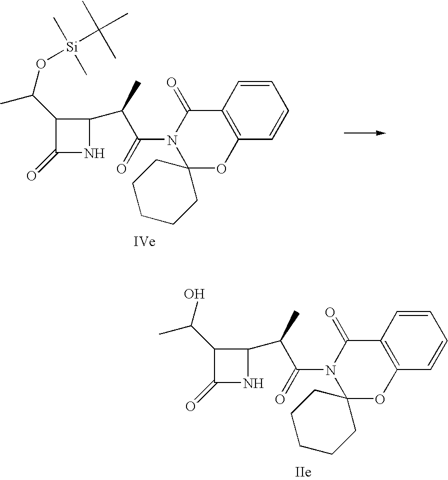

A compound of Formula IVe (5 g) was added to methanol (20 ml), followed by the addition of acetyl chloride (0.2 g). The reaction mixture was stirred for 2 hours at a temperature of about 25° C. and subsequently poured into a mixture of dichloromethane (50 ml) and water (50 ml). The dichloromethane layer was separated and concentrated, followed by recrystallization with a mixture of toluene and hexane to yield a compound of Formula IIe.

Yield: 3.2 g

¹H NMR (CDCl₃): 7.94 (m, 1H, ArH), 7.53 (m, 1H, ArH), 7.13 (m, 1H, ArH), 7.05 (m, 1H, ArH), 6.02 (br, 1H, NH), 4.19 (m, 1H, —CH—OH), 3.97 (m, 1H, H-4), 3.58 (m, 1H, —CH—CO), 3.27 (dd, 1H, H-3), 1.60-2.50 (m, OH, cyclohexyl), 1.34 (d, 3H, CH3), 1.26 (d, 3H, CH3)

### Example 10

### Preparation of Compound of Formula IIe:

Using the procedure set forth in Example 9, acetyl chloride was replaced with silica chloride (1.0 g) to obtain IIe. Thus, a compound of Formula IVe (5 g) was added to methanol (20 ml), followed by the addition of silica chloride (1 g). The reaction mixture was stirred for 2 hours at a temperature of about 25° C. and subsequently poured into a mixture of dichloromethane (50 ml) and water (50 ml). The dichloromethane layer was separated and concentrated, followed by recrystallization with a mixture of toluene and hexane to yield a compound of Formula IIe.

We claim:

1. A process for preparing a compound of Formula II 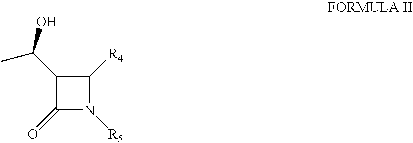
comprising:
desilylating a compound of Formula IV, 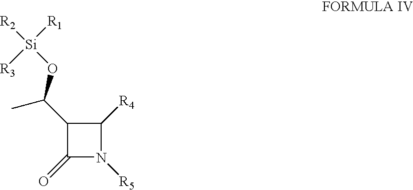
in the presence of one or more desilylating agents selected from acetyl chloride, silica chloride, thionyl chloride, oxalyl chloride or a mixture thereof to form a compound of Formula II,
wherein
P₁ is hydrogen or a carboxyl protecting group;
P₂ is hydrogen or an amino protecting group;
R₁, R₂ and R₃ are same or different and are each independently C₁₋₅ alkyl;
R₄ is selected from 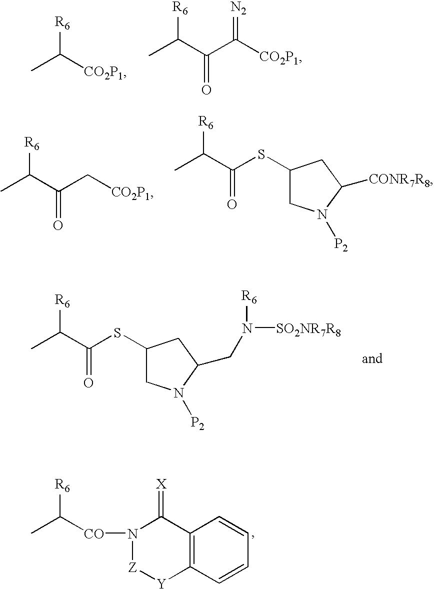
R₅ is hydrogen,
or R₄ and R₅ are joined together so as to form a resultant compound of Formula III; 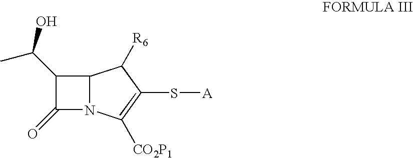
R₆ is hydrogen or C₁₋₅ alkyl;
R₇ and R₈ are same or different and are each hydrogen, C₁₋₅ alkyl, substituted or unsubstituted aryl, or substituted or unsubstituted heteroaryl;
X is oxygen atom or sulfur atom;
Y is oxygen atom, sulfur atom, substituted or unsubstituted methylene group or an imino group;
Z is a substituted or unsubstituted methylene group; and
A is selected from

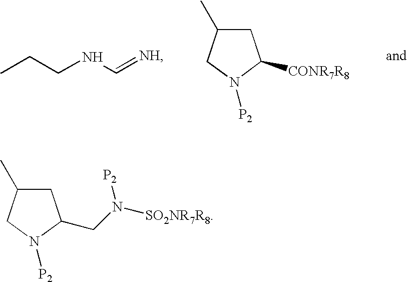

2. The process of claim 1, wherein the process is carried out in the presence of acetyl chloride.

3. The process of claim 1, wherein the process is carried out in the presence of silica chloride.

4. The process of claim 1, wherein the process is carried out in the presence of thionyl chloride.

5. The process of claim 1, wherein the process is carried out in the presence of oxalyl chloride.

6. The process of claim 1, wherein the process is carried out in the presence of one or more organic solvents selected from one or more of C₁₋₅ alkanols, aromatic hydrocarbons, halogenated hydrocarbons, ketones, esters, ethers or mixtures thereof.

7. The process of claim 6, wherein the one or more organic solvents is one or more C₁₋₅ alkanols.

8. The process of claim 1, wherein the compound of Formula II is isolated from the reaction mixture by layer separation or filtration.

9. The process of claim 8, wherein layer separation comprises adding a mixture of one or more miscible organic solvents and water after the compound of Formula IV is desilylated to form a layered mixture.

10. The process of claim 9, wherein the layered mixture is neutralized by adding one or more bases.

11. The process of claim 10, wherein the one or more bases are selected from one or more of metal hydroxides, metal carbonates, metal oxides, amines or mixtures thereof.

12. The process of claim 1, wherein the process is carried out at a temperature of about −20° C. to about 50° C.
